(19) United States
(12) Patent Application Publication
(10) Pub. No.: US 2006/0154948 A1
(43) Pub. Date: Jul. 13, 2006

(54) Treating allergic and inflammatory conditions

(75) Inventors: Melton B. Affrime, Warren, NJ (US); Christopher R. Banfield, High Bridge, NJ (US); Samir K. Gupta, East Brunswick, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: SCHERING CORPORATION

(21) Appl. No.: 11/033,919

(22) Filed: Jan. 12, 2005

(51) Int. Cl. A61K 31/473

(52) U.S. Cl. 514/290

(57) ABSTRACT

The use of desloratadine for the preparation of a medicament for treating and/or preventing an allergic and inflammatory condition of the skin or upper and lower airway passages in a pediatric patient and a pediatric pharmaceutical composition effective for such treating and/or preventing which comprises an effective amount of desloratadine and a pharmaceutically acceptable carrier are disclosed.

### BACKGROUND OF THE INVENTION

This invention relates to the use of desloratadine for the preparation of a medicament for treating and/or preventing allergic and inflammatory conditions in a pediatric patient and a pediatric pharmaceutical composition comprising an amount of desloratadine effective for such treating and/or preventing.

Loratadine is disclosed in U.S. Pat. No. 4,282,233 as a non-sedating antihistamine useful for treating allergic reactions in animals including humans. The recommended daily dose of loratadine is 10 mg, once daily, for adults and children, 12 years of age and older as well as for children, ages 6 to 11 (in the form of the syrup):.

Recent Food and Drug Administration (“FDA”) proposed regulations would require new drugs to include labeling on how such drugs could be used safely and effectively in pediatric populations. The FDA Modernization Act further addressed the need for improved information about the use of medicines in the pediatric population.

There is a need for a safe and clinically effective therapy to treat or prevent such allergic and inflammatory conditions of the skin and airway passages in pediatric patients.

### SUMMARY OF THE INVENTION

The present invention provides the use of desloratadine for the preparation of a medicament for treating and/or preventing an allergic and inflammatory condition of the skin or airway passages in a pediatric patient wherein the medicament comprises an effective amount of desloratadine and a pharmaceutically acceptable carrier.

The present invention also provides a pharmaceutical composition for treating and/or preventing an allergic and inflammatory condition of the skin or airway passages in a pediatric patient which comprises an effective amount of desloratadine and a pharmaceutically acceptable carrier.

The present invention provides a method of treating and/or preventing an allergic and inflammatory condition of the skin or airway passages in a pediatric patient in need of such treating and/or preventing which comprises administering an amount of desloratadine to the pediatric patient effective for such treating and/or preventing.

The present invention also provides a method of treating and/or preventing seasonal or perennial allergic rhinitis in a pediatric patient which comprises administering an amount of desloratadine to the pediatric patient effective for such treating and/or preventing.

The present invention provides a method of treating and/or preventing atopic dermatitis or urticaria in a pediatric patient in need of such which comprises administering an amount of desloratadine to the pediatric patient effective for such treating and/or preventing.

### BRIEF DESCRIPTION OF THE FIGURES

FIG. 1 graphically displays the variation over time (time zero to 96 hrs) of the mean plasma concentrations of desloratadine (ng/mL of plasma) following (I) a single 5 mL (2.5 mg) dose of desloratadine syrup (0.5 mg/mL) to pediatric volunteers ages 2-5 years and (ii) a single 10 mL (5.0 mg) dose of desloratadine syrup (0.5 mg/mL) to healthy adult volunteers ages 18 to 45 years.

FIG. 2 graphically displays the variation over time (time zero to 96 hrs) of the mean plasma concentrations of desloratadine (ng/mL of plasma) following (I) a single 10 mL (5 mg) dose of desloratadine syrup (0.5 mg/mL) to pediatric volunteers ages 6-11 years and (ii) a single 10 mL (5.0 mg) dose of desloratadine syrup (0.5 mg/mL) to healthy adult volunteers ages 18 to 45 years.

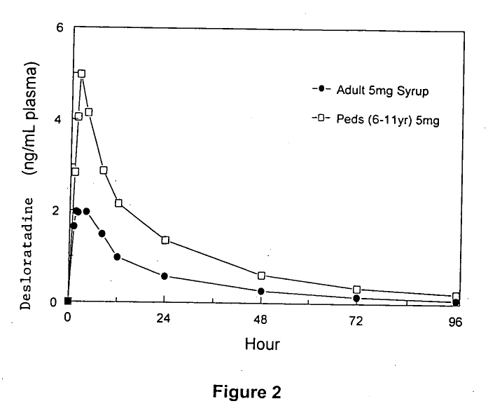

### DETAILED DESCRIPTION OF THE INVENTION

The phrase “allergic and inflammatory condition of the skin or airway passages” as used herein means those allergic and inflammatory conditions and symptoms found on the skin and in the upper and lower airway passages from the nose to the lungs. Typical allergic and inflammatory conditions of the skin or upper and lower airway passages include seasonal and perennial allergic rhinitis, non-allergic rhinitis, asthma including allergic and non-allergic asthma, sinusitis, colds (in combination with a NSAID, e.g., aspirin, ibuprofen or APAP) and/or a decongestant e.g. pseudoephedrine), dermatitis, especially allergic and atopic dermatitis, and urticaria and symptomatic dermographism as well as retinophathy, and small vessel diseases, associated with diabetes mellitus.

The amount of desloratadine effective for treating or preventing allergic and inflammatory conditions of the skin or airway passages will vary with the age, sex, body weight, growth and developmental changes as well as the severity of the allergic and inflammatory condition of the pediatric patient. Typically, the amount of desloratadine effective for treating or preventing such allergic and inflammatory conditions is in the range of about 2.5 mg/day for pediatric patients, ages 6 to less than 12 years, about 1.25 mg/day for pediatric patients, ages 2 to less than 6 years, and about 0.60 to about 0.70 mg/day, preferably about 0.63 mg/day, more preferably about 0.625 mg/day for pediatric patients, ages 6 months to less than 2 years, in single or divided doses, preferably a single daily dose in the form of a syrup.

Desloratadine is a non-sedating long acting histamine antagonist with potent selective peripheral H1-receptor antagonist activity. Following oral administration, loratadine is rapidly metabolized to descarboethoxyloratadine or desloratadine, a pharmacologically active metabolite. In vitro and in vivo animal pharmacology studies have been conducted to assess various pharmacodynamic effects of desloratadine and loratadine. In assessing antihistamine activity in mice (comparison of ED₅₀ value), desloratadine was relatively free of producing alterations in behavior alterations in behavior, neurologic or autonomic function. The potential for desloratadine or loratadine to occupy brain H1-receptors was assessed in guinea pigs following i.p. administration and results suggest poor access to central histamine receptors for desloratadine or loratadine.

In vivo studies also suggest that an inhibitory effect of desloratadine on allergic bronchospasm and cough can also be expected.

The clinical efficacy and safety of desloratadine has been documented in over 3,200 seasonal allergic rhinitis patients in 4 double-blinded, randomized clinical trials. The results of these clinical studies demonstrated the efficacy of desloratadine in the treatment of adult and adolescent patients with seasonal rhinitis.

Efficacy endpoints in all the studies were Total Symptom Score, Total Nasal Symptom Score, Total Non-nasal Symptom Score, and Health Quality of Life (HQOL) analysis in efficacy trials. Desloratadine (5 mg once daily) significantly reduced the total symptom scores (the sum of individual scores for rhinorrhea, sneezing, congestion/stuffiness, nasal itching, itchy/burning eyes, tearing, ocular redness, and itchy ears/palate). Desloratadine (5 mg) was significantly (p<0.01) more effective than placebo in reducing nasal symptoms. An important efficacy endpoint analyzed in the desloratadine studies is the AM NOW total symptom score. This parameter measures the total symptom relief by the patient after 24 hours before taking the next day dose. Statistically significant (p<0.05) reductions were maintained for the full 24 hour dosing interval over the entire dosage range There were no significant differences in the effectiveness of desloratadine (over the entire dosage range) across subgroups of patients defined by gender, age, or race. Desloratadine is particularly useful for the treatment and prevention of the nasal (stuffiness/congestion, rhinorrhea, nasal itching, sneezing) and non-nasal (itchy/burning eyes, tearing/watery eyes, redness of the eyes, itching of the ears/palate) symptoms of seasonal allergic rhinitis, including nasal congestion, in patients in need of such treating and/or preventing.

Clinical Study Designs

Study Treatments

Subjects were confined to the study site at least 12 hours prior to each treatment administration. In the morning of Day 1 following a 10 hour overnight fast, each subject received one of the following treatments based on his/her subject number and the study period: fasting (Treatment A) or did not eat again (Treatment B) until the 4-hour study procedures were completed, at which time lunch was served. Water was permitted throughout the fasting period except for 2 hours following treatment administration. The subjects remained awake and seated upright/ambulatory for 4 hours post-dose. A physician was present at the time of dosing and remained on site until at least 4 hours post-dose. Subjects were under medical supervision throughout their confinement at the study site. Each treatment administration was separated by at least a 7 day washout period.

Pharmacokinetics

Blood samples were collected for determination of the plasma pharmacokinetic profile of desloratadine. Fifteen milliliters (15 mL) of blood were collected just prior to drug administration (0 hour) and at pre-specified times after dosing in both periods. All blood samples were collected into heparin-containing tubes at the specified times. The blood samples were centrifuged within 30 minutes after collection for 20 minutes at approximately 4° C. and at approximately 3000 rpm. The plasma was separated and transferred into two separate appropriately labeled tubes, frozen to at least −20° C. and maintained in the frozen state until assayed for desloratadine content.

The plasma concentration data for desloratadine were used to estimate the following pharmacokinetic parameters using standard methodologies well known to those skilled in the art.

The major pharmacokinetic variables of interest were the plasma AUC and Cmax. All plasma samples were assayed for desloratadine concentrations using a validated method such as gas/liquid chromatography with a NP detector (GLC/NPD). The validation of the assay methods included documentation of its selectivity, limit of quantitation, linearity, precision and accuracy. The lower limit of quantitation (LOQ) of the assay was established at 0.1 ng/mL for desloratadine.

Safety Measurements Assessed

For safety evaluation, physical examinations, vital signs, electrocardiograms and clinical laboratory tests were conducted at screening and at the conclusion of the study. In addition, vital signs were monitored prior to treatment administration and daily during both treatment periods. Additional clinical laboratory tests and ECGs were obtained prior to dosing in each treatment period. The assessment, severity and relationship to treatment of adverse events were evaluated.

### Study No. 1

The objective of this study was to evaluate the effect of food on the bioavailability of desloratadine. These adult studies were designed to define the bioavailability/bioequivalence (BA/BE) relative to the tablet formulation, the effect of food on the pharmacokinetics following administration of the syrup formulation and the pharmacokinetic profile of desloratadine after single dose administration of the syrup to healthy male and female adult subject

### Single-Dose BA/BE Study

This was a Phase I, randomized, open-label, three-way crossover study in 30 healthy adult subjects (ages 18 to 45) who received a 5 mg desloratadine tablet and 10 mL of desloratadine syrup (0.5 mg/mL) under fasted conditions as well as following a high-fat, high-calorie breakfast on three separate occasions.

Subjects were confined at the study site at least 12 hours prior to each treatment (Day −1). In the morning of Day 1 following a 10 hour overnight fast, each subject received one of the following treatments based on his/her subject number and the study period:

| | |
|---|---|
| Treatment A: | One desloratadine (DL) 5 mg tablet |
| | administered after a 10 hour fast. |
| Treatment B: | Ten (10) mL of DL syrup (0.5 mg/mL) |
| | following a 10 hour fast |
| Treatment C: | Ten (10) mL DL syrup (0.5 mg/mL) |
| | administered immediately following a |
| | standardized high-fat, high caloric |
| | breakfast. |

Subjects randomized to receive the standardized high-fat, high caloric breakfast (Treatment C) consumed the prescribed meal in a 20-minute period prior to drug administration and received the appropriate dose of desloratadine within 5 minutes after completing the breakfast.

Study Population/Inclusion Criteria/Exclusion Criteria

Inclusion Criteria:
Subjects were males or females between the ages of 18 and 45 years inclusive, and had a Body Mass Index (BMI) between 19-27.
Clinical laboratory tests (CBC, blood chemistries, urinalysis) were within normal limits or clinically acceptable to the Investigator/Sponsor.
Drug screen for drugs with a high potential for abuse were negative at screening and on admission to the study site.
Subjects were free of any clinically significant disease that required a physician's care and/or may have interfered with study evaluations, procedures or participation.
Subject gave written informed consent (prior to any study-related procedures being performed) and were willing to adhere to restrictions and examination schedules.
Subjects had a normal or clinically acceptable physical examination and ECG.

Exclusion Criteria:
Subjects who had a history of any clinically significant local or systemic infectious disease within four weeks prior to initial treatment administration.
Subjects who did not comply with the requirement that he or she should not have used any drugs (except acetaminophen) within 14 days prior to the study nor alcohol or xanthine-containing substances with 72 hours prior to study drug administration.
Subjects who had participated in a clinical trial of any investigational drug within 30 days prior to the start of the study.
Subjects who were, or were known to be former, narcotic addicts or alcoholics.
Subjects who were positive for hepatitis B surface antigen or hepatitis C antibody.
Subjects who were positive for HIV antibodies.
Subjects who had a clinically significant history of food or drug allergy.
Subjects who had a known allergy or intolerance to loratadine.
Subjects who smoked, used tobacco products or used an adjunct to smoking cessation within the past 6 months (positive urine test).
Females who were not surgically sterilized or were considering reversal of their surgical sterilization or were not at least 1 year post-menopausal.
Females who had a positive urine pregnancy test at screening or on admission to the study site.
Females who were lactating.
   Study Treatments

Subjects were confined to the study site at least 12 hours prior to each treatment administration. In the morning of Day 1 following a 10-hour overnight fast, each subject received one of the following doses.

Each dose was administered with 180 mL (6 fl oz) of non-carbonated room temperature water. The tablet was swallowed whole, not chewed or crushed. After dosing, the oral cavity will be inspected to assure that the subject had swallowed the tablet/syrup. For subjects randomized to Treatment B or Treatment C the study medication was administered by having the volunteer drink the entire 10 mL of desloratadine syrup, followed by two 10 mL tap water rinses of the dose container (i.e., oral syringe, etc.) to ensure complete dose intake. Subjects continued fasting (Treatment A and B) or did not eat again (Treatment C) until the 4-hour study procedures were completed, at which time lunch was served. Water was permitted throughout the fasting period except for 2 hours following treatment. The subjects remained awake and seated upright/ambulatory for 4 hours post-dose.

All subjects were confined to the study site until the 120-hour blood samples, vital signs and laboratory tests were obtained. No strenuous physical activity was permitted, and the subjects were not allowed visitors while they were confined to the study site. A washout period of at least 14 days separated each period of the study.

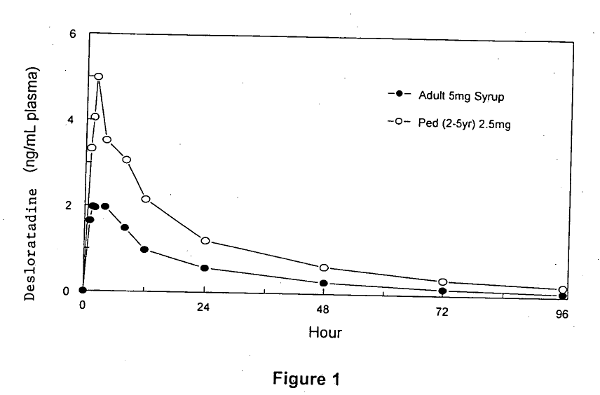

The mean pharmacokinetic profiles of desloratadine following single dose administration of the syrup formulation under fasted conditions are illustrated in FIG. 1.

### Study No. 2

### Single-Dose Pharmacokinetics in Pediatric Subjects (≧2 to <6 Years Old)

The objective of this open label study was to characterize the pharmacokinetic profile of desloratadine and 3-OH desloratadine following a single dose of 5 mL (2.5 mg) desloratadine syrup (0.5 mg/mL) administered orally to healthy pediatric subjects ranging in age from ≧2 to <6 years. These pediatric subjects were found to have normal or clinically acceptable laboratory tests be free of any clinically significant disease and to have normal or clinically acceptable ECGs

A total of 18 healthy pediatric subjects (12 males and 6 females) with at least 4 subjects in the following age groups: ≧2 but <3, ≧3 but <4, ≧4 but <5, ≧5 but <6 were enrolled and successfully completed this open-label, single-center study. In this study, each subject received a single 5 mL (2.5 mg) dose of desloratadine syrup (0.5 mg/mL) administered orally.

Subjects were screened within 3 weeks of dosing, and those who met the entry criteria were confined to the study center within 24 hours prior to dosing. Upon confinement, the clinical laboratory safety tests performed at Screening were repeated for each subject. The next morning all subjects received the study medication. Vital signs were obtained daily. Blood samples were collected at pre-specified times before and after dosing for safety and pharmacokinetic evaluations. Subjects were continually observed and questioned throughout the study for the possible occurrence of adverse events. Subjects were also instructed to report any unusual experiences or discomfort. No strenuous physical activity was permitted, and the subjects were not allowed visitors (besides the parents or legal guardians) while they were confined to the study site. Following the 24-hour study-related procedures for safety and pharmacokinetic evaluations, subjects were dismissed from the study site. They returned to the study site on Days 3, 4 and 5 for the 48-hour, 72-hour and 96-hour study-related procedures. Following completion of all study-related procedures on Day 5, subjects were discharged from the study.

The mean plasma concentration-time (0-96 hrs) profiles of desloratadine following administration of desloratadine to pediatric subjects ≧2 to <6 years old and adults 18 to 45 years old are illustrated in FIG. 1.

The derived pharmacokinetic parameters are provided in Table 1.

TABLE 1
Mean Pharmacokinetic Parameters
The Mean (% CV) Pharmacokinetic Parameters of Desloratadine
Following Oral, Single-Dose Administration of 5.0 & 2.5 mg
of Desloratadine under Fasted Conditions:

| | Mean (% CV)¹ | | |
|---|---|---|---|
| | | Cmax | AUC(96) |
| Syrup Data | N | (ng/mL) | (ng · hr/mL) |
| Adults(5 mg) | 30 | 2.19 | 45.2 |
| Pediatrics(≧2 to <6 yr, | 18 | 5.36 | 98.6 |
| 2.5 mg) | | | |

¹% CV is percent coefficient of variation, which is a relative measure of variability. See Steele and Torrie, “Principles and Procedures of Statistics”, (1980) 2^{nd} Edition, McGraw-Hill, NY, at page 27.

Single oral doses of 5 mL (2.5 mg) of desloratadine syrup administered to healthy pediatric volunteers ≧2 to <6 years of age was safe and well tolerated. To obtain similar systemic exposure in pediatric subjects (≧2 to <6 years of age) as found in adults administered a 5 mg dose, the 2.5 mg dose should be reduced by 50% to 1.5 mg (See Study No. 4).

### Study No. 3

### Single-Dose Pharmacokinetics in Pediatric Subjects (≧6 to <12 Years Old)

This open-label study in 18 healthy pediatric subjects was designed to characterize the pharmacokinetic profile of desloratadine and 3-OH desloratadine following a single 2.5 mg (5 mL) dose of desloratadine syrup administered orally to healthy pediatric volunteers ranging in age from ≧6 to <12 years. These pediatric patients were found to have normal or clinically acceptable laboratory tests be free of any clinically significant disease and to have normal or clinically acceptable ECGs

The objective of this study was to characterize the pharmacokinetic profile of desloratadine following a single dose of 10 mL (5 mg) desloratadine syrup (0.5 mg/mL) administered orally to healthy pediatric subjects ranging in age from ≧6 to <12 years.

A total of 18 healthy pediatric subjects (9 males and 9 females) with at least 3 subjects in the following age groups: ≧6 but <7, ≧7 but <8, ≧8 but <9, ≧9 but <10, ≧10 but <11, and ≧11 but <12 were enrolled and successfully completed this open-label, single-center study. In this study, each subject received a single 10 mL (5 mg) dose of desloratadine syrup (0.5 mg/mL) administered orally.

Subjects were screened within 3 weeks of dosing, and those who met the entry criteria were confined to the study center within 24 hours prior to dosing. Upon confinement, the clinical laboratory safety tests performed at Screening were repeated for each subject. The next morning all subjects received the study medication. Vital signs were obtained daily. Blood samples were collected at pre-specified times before and after dosing for safety and pharmacokinetic evaluations. Subjects were continually observed and questioned throughout the study for the possible occurrence of adverse events. Subjects were also instructed to report any unusual experiences or discomfort. No strenuous physical activity was permitted, and the subjects were not allowed visitors (besides the parents or legal guardians) while they were confined to the study site. Following the 24-hour study-related procedures for safety and pharmacokinetic evaluations, subjects were dismissed from the study site. They returned to the study site on Days 3, 4 and 5 for the 48-hour, 72-hour and 96-hour study-related procedures. Following completion of all study-related procedures on Day 5, subjects were discharged from the study.

The mean plasma concentrations of desloratadine following administration of (i) a single 5 mL (2.5 mg) dose of desloratadine syrup (0.5 mg/mL) to pediatric subjects (ages ≧6 to <12) and (ii) a single 10 mL (5.0 mg) dose of desloratadine syrup (0.5 mg/mL) to adult subjects (ages 18 to 45).

The derived pharmacokinetic parameters are provided in Table 2

TABLE 2
Mean Pharmacokinetic Parameters
The Mean (% CV) Pharmacokinetic Parameters of Desloratadine
Following Oral, Single-Dose Administration of 5.0 mg of
Desloratadine under Fasted Conditions:

| | Mean (% CV)¹ | | |
|---|---|---|---|
| | | Cmax | AUC(96) |
| Syrup Data | N | (ng/mL) | (ng · hr/mL) |
| Adults(5 mg) | 30 | 2.19 | 45.2 |
| Pediatrics(≧6 to <12 yr, | 18 | 5.3 | 101 |
| 5.0 mg) | | | |

¹% CV is percent coefficient of variation, which is a relative measure of variability. See Steele and Torrie, “Principles and Procedures of Statistics”, (1980) 2^{nd} Edition, McGraw-Hill, NY, at page 27.

Single oral doses of desloratadine syrup administered to healthy pediatric subjects ≧6 to <12 years of age was safe and well tolerated. To obtain similar systemic exposure in pediatric subjects (≧6 to <12 years of age) as in adults administered 5 mg, the dose should be reduced by 50% to 2.5 mg (See Study No. 5).

Based on the findings of the studies pharmacokinetic studies will be repeated in pediatric subjects between the ages of ≧2 to <6 years and ≧6 to <12 years.

### Study No. 4

### Single Dose Pharmacokinetics of Desloratadine Syrup in Healthy Pediatric Volunteers 2-5 Years of Age

Study Objective:

The objective of this open label study will be to characterize the pharmacokinetic profile of desloratadine following a single dose of 2.5 mL (1.25 mg) desloratadine syrup (0.5 mg/mL) administered orally to healthy pediatric subjects ranging in age from ≧2 to <6 years. These pediatric subjects selected for inclusion into this open label study should have normal or clinically acceptable laboratory tests, be free of any clinically significant disease and have normal or clinically acceptable ECGs

Study Design:

A total of eighteen (18) healthy male or female pediatric volunteers—with at least three subjects at each age stratification-will receive a single dose of 2.5 mL (1.25 mg) of desloratadine syrup (0.5 mg/mL) administered orally. The protocol of Study No. 2 will be followed

Study Endpoints:

The following pharmacokinetic parameters will be obtained from the resulting desloratadine concentration-time profiles:
Area under the concentration-time curve (AUC_{0-∞}, AUC₀₋ₜ)
Peak concentration (Cₘₐₓ)
Time to peak concentration (Tₘₐₓ)

### Study No. 5

### Single Dose Pharmacokinetics of Desloratadine Syrup in Healthy Pediatric Volunteers ≧6 to <12 Years of Age

Study Objective: The objective of this open label study will be to characterize the pharmacokinetic profile of desloratadine and 3-OH desloratadine following a single dose of 5.0 mL (2.5 mg) desloratadine syrup (0.5 mg/mL) administered orally to healthy pediatric subjects ranging in age from ≧6 to <12 years. These pediatric patients selected for inclusion into this open label study should have normal or clinically acceptable laboratory tests, be free of any clinically significant disease and have normal or clinically acceptable ECGs

Study Design:

A total of eighteen (18) healthy male or female pediatric volunteers ages from ≧6 to <12 years—with at least three subjects at each age stratification will receive a single dose of 5 mL (2.5 mg) desloratadine syrup (0.5 mg/mL) administered orally. The protocol of Study No. 3 will be followed

Study Endpoints:

The following pharmacokinetic parameters will be obtained from the resulting desloratadine concentration-time profiles:
Area under the concentration-time curve (AUC_{0-∞}, AUC₀₋ₜ)
Peak concentration (Cₘₐₓ)
Time to peak concentration (Tₘₐₓ)

U.S. Pat. No. 4,659,716 discloses methods of making desloratadine, pharmaceutical compositions containing it and methods of using desloratadine and pharmaceutical compositions containing it to treat allergic reaction in mammals.

U.S. Pat. No. 5,595,997 discloses pharmaceutical compositions containing desloratadine and methods of using desloratadine for treating and preventing various disease states, e.g., allergic rhinitis.

Desloratadine is available from Schering Corporation, Kenilworth, N.J.

The desloratadine syrup (0.5 mg/ml) is disclosed in International Patent Application PCT/US99/10469 having an international application date of 27 May 5, 1999

The pharmaceutical compositions of desloratadine can be adapted for any mode of administration e.g., for oral, parenteral, e.g., subcutaneous (“SC”), intramuscular (“IM”), and intraperitoneal (“IP”), topical or vaginal administration or by inhalation (orally or intranasally). Preferably desloratadine is administered orally.

Such pharmaceutical compositions may be formulated by combining desloratadine or an equivalent amount of a pharmaceutically acceptable salt thereof with a suitable, inert, pharmaceutically acceptable carrier or diluent that may be either solid or liquid. Desloratadine may be converted into the pharmaceutically acceptable acid addition salts by admixing it with an equivalent amount of a pharmaceutically acceptable acid. Typically suitable pharmaceutically acceptable acids include the mineral acids, e.g., HNO₃, H₂SO₄, H₃PO₄, HCl, HBr, organic acids, including, but not limited to, acetic, trifluoroacetic, propionic, lactic, maleic, succinic, tartaric, glucuronic and citric acids as well as alkyl or arylsulfonic acids, such as p-toluenesulfonic acid, 2-naphthalenesulfonic acid, or methanesulfonic acid. The preferred pharmaceutically acceptable salts are trifluoroacetate, tosylate, mesylate, and chloride. Desloratadine is more stable as the free base than as an acid addition salt and the use of the desloratadine free base in pharmaceutical compositions of the present invention is more preferred.

Solid form preparations include powders, tablets, dispersible granules, capsules, cachets and suppositories. The powders and tablets may be comprised of from about 5 to about 95 percent active ingredient. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, syrups suspensions and emulsions.

Preferably, the pharmaceutical preparation is in a unit dosage form. In such form, the preparation is subdivided into suitably sized unit doses containing appropriate quantities of the active component, e.g., an effective amount to achieve the desired purpose.

1. (canceled)

2) A method treating and/or preventing allergic and inflammatory conditions of the skin or airway passages in a pediatric patient in need of such treating and/or preventing which comprises administering an amount of desloratadine to the pediatric patient effective for such treating and/or preventing.

3) The method of claim 2 wherein the pediatric patient is 6 to less than 12 years of age and the amount of desloratadine is about 2.5 mg/day.

4) The method of claim 2 wherein the pediatric patient is 2 to less than 6 years of age and the amount of desloratadine is about 1.25 mg/day.

5) The method of claim 2 wherein the pediatric patient is about 6 months to less than 2 years of age and the amount of desloratadine is about 0.60-0.70 mg/day.

6) The method of claim 2 wherein the allergic reaction is season allergic rhinitis, perennial allergic rhinitis, atopic dermatitis, urticaria or allergic asthma.

7) A method of treating and/or preventing seasonal or perennial allergic rhinitis in a pediatric patient which comprises administering an amount of desloratadine to the pediatric patient effective for such treating and/or preventing.

8) The method of claim 7 wherein the pediatric patient is about 6 to less than 12 years of age and the amount of desloratadine is about 2.5 mg/day.

9) The method of claim 7 wherein the pediatric patient is about 2 to less than 6 years of age and the amount of desloratadine is about 1.25 mg/day.

10) The method of claim 7 wherein the pediatric patient is about 6 months to less than 2 years of age and the amount of desloratadine is about 0.60-0.70 mg/day.

11) A method of treating and/or preventing atopic dermatitis or urticaria in a pediatric patient in need of such which comprises administering an amount of desloratadine to the pediatric patient effective for such treating and/or preventing.

12) The method of claim 11 wherein the pediatric patient is about 6 to less than 12 years of age and the amount of desloratadine is about 2.5 mg/day.

13) The method of claim 11 wherein the pediatric patient is about 2 to less than 6 years of age and the amount of desloratadine is about 1.25 mg/day.

14) The method of claim 11 wherein the pediatric patient is about 6 months to less than 2 years of age and the amount of desloratadine is about 0.60-0.70 mg/day.
